(19) United States
(12) Patent Application Publication
(10) Pub. No.: US 2005/0166918 A1
(43) Pub. Date: Aug. 4, 2005

(54) Apparatus for nebulising a liquid, in particular for aerosol therapy

(76) Inventor: Andrea Trombi, Parma (IT)

Correspondence Address: ARENT FOX PLLC, 1050 CONNECTICUT AVENUE, N.W., SUITE 400, WASHINGTON, DC, 20036, US

(21) Appl. No.: 10/512,072

(22) PCT Filed: Oct. 28, 2002
(86) PCT No.: PCT/IT02/00681

(30) Foreign Application Priority Data
May 28, 2002 (IT) PR2002A000026

(51) Int. Cl. A61M 15/00; A61M 16/10

(52) U.S. Cl. 128/203.12; 128/203.16

(57) ABSTRACT

Nebulising apparatus (1) for aerosol therapy comprising a nebuliser ampoule (2) provided with at least an opening (4;5) for aspirating and/or expelling air from/to the environment and with a mouthpiece (3) for dispensing a nebulised medical product. The apparatus is provided with a valve (6) for regulating a flow of air into and/or out of the ampoule (2) and it comprises: a shutter (8) able to move between an operative configuration corresponding to an obstruction of said opening (4;5) and an operative configuration of consent to the passage of the flow of air; a ring (9) connected to the shutter (8) to anchor it to a tubular portion (2a) of the ampoule (2); a plurality of deformable connecting elements (10) between the ring (9) and the shutter (8) to allow the shutter (8) to move between the aforesaid operative configurations.

### TECHNICAL FIELD AND BACKGROUND ART

The present invention relates to an apparatus for nebulising a liquid, in particular for aerosol therapy.

The apparatus comprises a nebulising ampoule provided with at least an opening for drawing and/or expelling air from/to the environment and is provided with a mouthpiece to dispense a nebulised medical product. The apparatus further comprises a valve for regulating a flow of air entering and/or exiting the ampoule, said valve being positioned in correspondence with the aforesaid opening.

As is well known, apparatuses for nebulising are used in particular in the field of aerosol therapy, i.e. of the therapeutic treatment of symptoms of the respiratory track, such as asthmatic or bronchial symptoms. Said therapeutic system provides for the generation of an aerosol, i.e. of a dispersion or nebulisation of appropriate medical liquids that act through the inhalation of the medical liquid itself.

Such apparatuses are widely used, especially in the case of paediatric therapies, and are provided in different formats able to meet different users' requirements. More specifically, nebulising apparatuses can also be constructed in portable formats, so that the user can have the necessary medicine available at any time, especially in the case of ailments entailing frequent or unpredictable respiratory crises, such as asthmatic ailments.

Pneumatic nebulising apparatuses also exist, so defined because they comprise a compressor that aspirates air from the environment and sends it to a nebulising ampoule containing the medical liquid.

The compressor is generally housed in a rigid case, made of instance of plastic material, which incorporates the inlets and outlets of the aspiration and delivery conduits that come from the compressor itself. In use, mainly with apparatuses for home use, the rigid case containing the compressor is usually set down on a plane whilst the nebulising ampoule is located in proximity to the user's face and is connected to the inlet of the delivery conduit by means of a flexible pipeline.

The compressor can comprise a header incorporating both the aspiration conduit and the delivery conduit, interfacing directly with the exterior by means of intakes obtained directly on the header itself and destined to be adapted to the profile of the rigid containment case.

Some pneumatic nebulisation apparatuses are provided with a so-called supplementary, or secondary, channel, provided with an inlet through which ambient air enters by Venturi effect and because of the aspiration provided by the user during inspiration.

The flow of air of the secondary channel allows a better nebulisation of the medical product, in terms of quantity and quality of the generated spray.

During the expiration phase, the air breathed out by the user is expelled from the apparatus by means of an outlet.

Normally, both the inlet of the secondary channel and the outlet are provided with a valve, able to move between an opened position and a closed position to guarantee that the flow of air inside the apparatus is correctly directed, both during inspiration and during expiration. In particular, said valves are usually made of highly deformable plastic material and are actuated directly by the flow of air that impacts thereon.

A possible known embodiment of said valves provides for the use of a rubber tab that has a first end fastened in correspondence with a mouth of a conduit and a second end free, in such a way as to cover the mouth itself and to be able to be lifted directly by a flow of air.

In accordance with a second type of prior art, valves for nebulisation apparatuses exist which are constituted by a ring whereto are peripherally welded a plurality of reeds, in such a way that a free end thereof (opposite to the welded one) can move under the direct actuation of the flow of air that traverses the valve. The ring provided with the reeds is inserted inside a pair of flanges, usually made of plastic material, mutually coupled by means of interference.

The valve types for nebulisation apparatuses briefly described above have some important drawbacks.

First of all, such valves, although made of a deformable material, are at times hard to operate and to open them and/or close them a flow of air with sufficient pressure is required. This has repercussions on the user, since (s)he sets the air aspiration and expulsion pressure during inspiration and expiration.

An additional drawback is represented by the fact that the aforementioned valves do not assure an effective closure, especially those that are normally closed in the resting configuration. In addition to compromising the correct operation of the nebulisation apparatus, this drawback entails inevitable wastage of medical product.

### DISCLOSURE OF INVENTION

An aim of the present invention is to eliminate the aforesaid drawbacks by making available an apparatus for nebulising a liquid, in particular for aerosol therapy, which is provided with valves that oppose a minimum resistance to opening and/or closing during inspiration and/or expiration by a user.

Another aim of the present invention is to propose a nebulising apparatus that is provided with valves which are normally closed in resting configuration and which assure a perfect seal, in order to limit wastage of medical product and assure the proper operation of the apparatus.

Said aims are fully achieved by the apparatus for nebulising a liquid, in particular for aerosol therapy, of the present invention, which is characterised by the content of the claims set out below and in particular in that the valve for regulating a flow of air is of the type comprising:
a shutter able to move between an operative blocking configuration, corresponding to an obstruction of the opening, and an operative configuration consenting to the passage of the flow of air;
a ring connected to the shutter to anchor it to a tubular portion of the ampoule, said tubular portion being positioned in correspondence with the opening;
a plurality of deformable connecting elements between the ring and the shutter to allow the shutter to move from said operative blocking configuration to said operative consent configuration and vice versa, said movement being directly caused by the flow of air entering and/or exiting the ampoule.

### BBEST MODE FOR CARRYING OUT OF THE INVENTION

This and other aims will become more readily apparent from the description that follows of a preferred embodiment illustrated, purely by way of non limiting example, in the accompanying drawing table, in which:

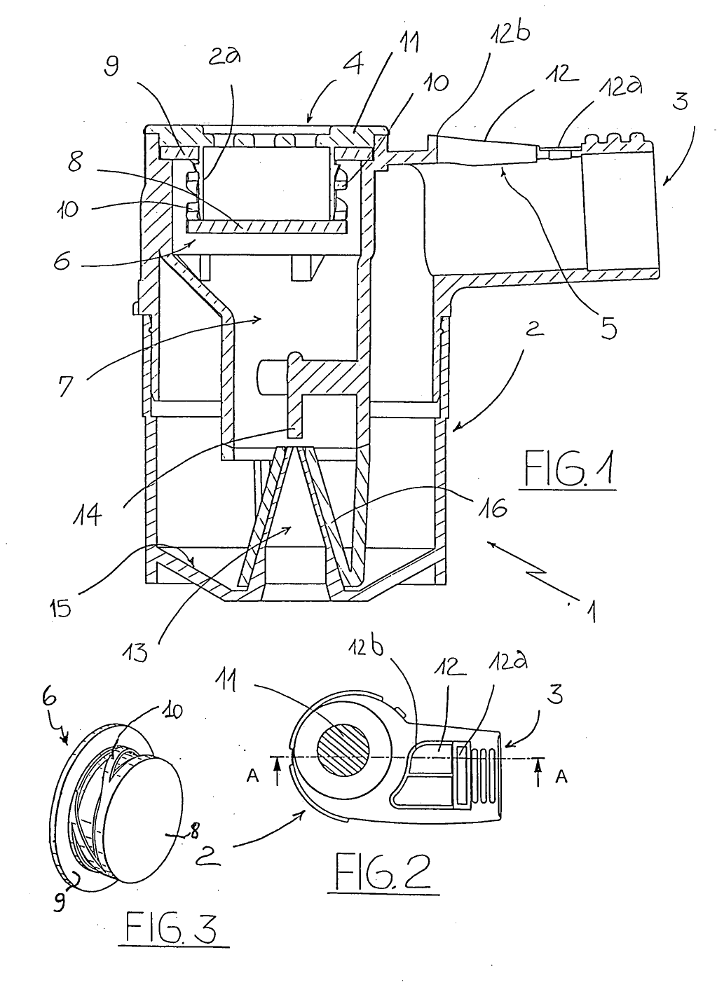

FIG. 1 shows a partially sectioned front view of an apparatus for nebulising a liquid according to the present invention;

FIG. 2 shows a top view of the apparatus shown in FIG. 1;

FIG. 3 shows a perspective view of the valve for regulating the flow of air.

With reference to the figures, the apparatus for nebulising a liquid in accordance with the present invention is globally indicated with the number 1 and comprises a nebuliser ampoule 2 provided with a mouthpiece 3 for dispensing a nebulised medical product directly into a user's oral cavity.

The nebuliser ampoule 2 is provided with a first opening 4 for aspirating air from the environment, during the inspiration phase, and with a second opening 5 for expelling air into the environment, during the expiration phase. The apparatus 1 comprises a valve 6 for regulating a flow of air entering the nebuliser ampoule 2. This valve is positioned in correspondence with the aforesaid opening 4 necessary to aspirate air from the environment and it is preferably associated to a so-called supplementary, or secondary, channel 7 of the nebuliser ampoule.

The flow regulating valve 6 is of the type comprising a shutter 8 able to move between an operative blocking configuration corresponding to an obstruction of the opening 4 (as shown in FIG. 1) and an operative configuration corresponding to an obstruction of the opening 4 (as shown in FIG. 1) and an operative configuration (not shown herein) of consent to the passage of the flow of air.

The shutter 8 is anchored to a tubular portion 2a of the nebuliser ampoule 2 by means of a ring 9 connected to the shutter by means of a plurality of deformable elements 10 which elements, given their deformability, allow the shutter 8 to move from the operative blocking configuration to the operative consent configuration and vice versa. In particular, said movement is directly caused by the vacuum generated on the shutter 8 by the user. In the illustrated embodiment, the deformable elements 10 are spiral shaped and have a first end fastened peripherally to the shutter 8 and a second end fastened to the ring 9.

The shutter 8, the ring 9 and the deformable elements 10 are preferably obtained in a single body and are made of polymeric material, typically rubber.

The apparatus 1 further comprises a protective element 11, holed and positioned in correspondence with the opening 4, to avoid introducing foreign bodies in the nebuliser ampoule 2, preventing any damage to the valve 6.

In the illustrated embodiment, the apparatus 1 further comprises a second shutter 12 to cover the second opening 5, necessary to expel into the environment air exhaled by a user. In the illustrated embodiment, the second shutter 12 is constituted by a deformable reed-like body, typically a rubber tab, having an end 12a fastened in correspondence with the opening 5 and an end 12b free to move away from said opening to uncover it at least partially and allow air to escape.

In accordance with an embodiment variation not shown herein, it is possible to use a second valve 6 coupled to an opening necessary to expel the air exhaled by the user.

The apparatus 1 comprises a compressor (not shown), which sends air to the nebuliser ampoule 2 by means of a primary delivery channel 13 which preferably has conical shape. As soon as the air coming from the compressor impacts against an activator element 14, inside the ampoule such a turbulence is generated as to create a sufficient vacuum to aspirate the medical liquid through a series of channels (not shown herein) obtained directly on a substantially conical element 16, positioned in correspondence with the primary channel 13.

The supplementary conduit 7 enables to increase the nebulisation of medical products, and also allows a coarse selection of the particles present in the spray. In particular, particles of greater size are forced to settle on a bottom 15 of the nebuliser ampoule 2 and therefore only the particles having the optimal dimensions to render the aerosol therapy more effective come out of the dispensing mouthpiece 3.

During inspiration, the vacuum generated inside the ampoule 2 draws air in from the environment, forcing the shutter 8 to move away from the opening 4. At the end of the inspiration phase, the shutter moves to the resting configuration, i.e. to obstruct the opening 4, reducing spray formation. In this way, during the expiration phase, the wastage of medical product that exits the opening 5, borne by the air exhaled by the user, is limited. The exhaled air exits thanks to the rising of the second shutter 12 which uncovers, at least partially, the second opening 5 of the nebuliser ampoule. The rising of the second shutter 12 is made possible by the pressure exerted by the air exhaled by the user.

The invention achieves important advantages.

First of all, the presence of the valve 6 allows considerably to reduce the effort required from the user during the inspiration phase and possibly also during the expiration phase, if an additional valve 6 is adopted in correspondence with the opening 5, necessary to expel the air exhaled by the user. Moreover, with a reduced effort on the user's part, it is possible to generate such a displacement of the shutter 8 as to maximise the area of the passage section of the air flow.

Another advantage is provided by the fact that the presence of the deformable elements 10 enables the valve to operate in immediate fashion, opening it and closing it in very short time intervals. This allows to optimise the operation of the apparatus 1, reducing in particular the wastage of medical product.

Advantageously, such an apparatus is simple and economical to construct.

1. Apparatus (1) for nebulising a liquid, in particular for aerosol therapy, of the type comprising:
a nebulising ampoule (2) provided with at least an opening (4; 5) for aspirating and/or expelling air from/to the environment and with a mouthpiece (3) for dispensing a nebulisation of medical product;
a valve (6) for regulating a flow of air into and/or out of the ampoule (2), said valve (6) being positioned in correspondence with said opening (4; 5), and comprising a shutter (8) able to move between an operative blocking configuration corresponding to an obstruction of said opening (4; 5) and an operative configuration of consent to the passage of the flow of air a ring (9) connected to the shutter (8) to anchor it to a tubular portion (2a) of the ampoule (2), said tubular portion (2a) being positioned in correspondence with the opening (4; 5) and a plurality of deformable connecting elements (10) between the ring (9) and the shutter (8) to allow said shutter (8) to move from said operative blocking configuration to said operative configuration of consent and vice versa, said movement being directly caused by an inspiration and/or expiration phase by a user, characterised in that the deformable elements (10) are spiral shaped and have a first end fastened peripherally to the shutter (8) and a second end fastened to the ring (9).

2. (canceled)

3. Apparatus as claimed in claim 1, characterised in that it comprises a holed protective element (11) positioned in correspondence with the opening (4; 5) to prevent the introduction of foreign bodies into the ampoule (2), preventing any damage to the valve (6).

4. Apparatus as claimed in claim 1, characterised in that it comprises a second shutter (12) to cover an opening (5) of the ampoule (2) necessary to expel to the environment air exhaled by a user, said second shutter (12) being a deformable reed-like body having an end (12a) fastened in correspondence with the opening (5) and an end (12b) that is free to move away from the opening (5) to uncover it at least partially and allow the escape of air.

5. Apparatus as claimed in claim 1, characterised in that the valve (6) is associated with a so-called supplementary, or secondary, channel (7) of the nebuliser ampoule (2).
